(19) United States
(12) Patent Application Publication
(10) Pub. No.: US 2003/0235837 A1
(43) Pub. Date: Dec. 25, 2003

(54) High resolution typing system for pathogenic E. coli

(76) Inventors: Paul Keim, Flagstaff, AZ (US); Christine Keys, College Park, MD (US)

Correspondence Address: QUARLES & BRADY LLP, RENAISSANCE ONE, TWO NORTH CENTRAL AVENUE, PHOENIX, AZ, 85004-2391, US

(21) Appl. No.: 10/317,444

(22) Filed: Dec. 11, 2002

Related U.S. Application Data
(60) Provisional application No. 60/339,687, filed on Dec. 11, 2001.

(51) Int. Cl. C12Q 1/68; C07H 21/04; C12P 19/34

(52) U.S. Cl. 435/6; 435/91.2; 536/23.7

(57) ABSTRACT

Variable number tandem repeat (VNTR) sequences have been identified in the genome of certain E. coli O157:H7 strains. It has been discovered that the VNTR sequences exhibit length polymorphism at different loci. A sub-typing system based on multiloci size analysis of VNTR is the basis of the novel molecular sub-typing system of the present invention.

### CROSS REFERENCE

This application claims priority from U.S. Provisional Patent Application Serial No. 60/339,687, filed Dec. 11, 2001, the disclosure of which is incorporated by reference herein in its entirety.

### FIELD OF INVENTION

This invention relates generally to molecular sub-typing of bacteria by genetic analysis of variable number tandem repeat (VNTR) sequences. More specifically, the invention is directed to a system for DNA sub-typing of pathogenic E. coli by multiple-locus variable number tandem repeat analysis (MLVA) and to an 15 epidemiological database constructed 15 from data generated by the system.

### BACKGROUND

In the light of recent meat-related food scares, public concern about food safety caused by E coli contamination continues to escalate. Disease incidence associated with the O157:H7 pathogenic strains has been on the rise since the 1980s when it was first associated with hemorrhagic colitis (18). Many outbreaks have been described subsequently worldwide, but in the United States it is well known for its appearance in a Washington State fast food chain outbreak (9). Since this infamous media introduction to the public, it has been associated with several very large packaged meat recalls (3, 4), outbreaks in daycare centers (1) and has even been associated with water-borne infections at an amusement park (20, 5, 12).

Crucial epidemiological links between outbreaks of E. coli infections at disparate times and places would help prevent the spread of disease. The challenge is to identify the strain and correlate it with source. Molecular typing has long been a part of pathogen identification and control. Traditionally, serotyping has been used to identify important cellular components associated with virulence. Newer approaches include multilocus enzyme electrophoresis, DNA typing, and ribotyping. Comparative gene sequencing such as multiple-locus sequence typing (MLST) has been used to distinguish among both species and strains and is useful in subtyping those bacteria presenting sufficient nucleotide diversity.

Currently, the most widespread approach to subtyping enteric pathogens is pulse field gel electrophoresis (PFGE) detection of restriction fragment length polymorphisms (19). Pulsed-field gel electrophoresis (PFGE) can resolve very large and sometimes polymorphic DNA restriction fragments. PFGE data is currently generated by hundreds of laboratories across the U.S. that contribute to PulseNet, a database established for epidemiological monitoring of outbreaks (19). However, PFGE is a cumbersome technology that cannot easily handle very large sample sets. Nor is PFGE data well suited for database comparisons due to the continuous nature of fragment sizes and has limited discrimination capacity for closely related isolates. Moreover, PFGE data sets are not easily standardized for transfer throughout the public health community. Yet PFGE is a “universal” technology that works on any bacteria without requiring prior genomic information for primer design. For at least the near future, the PFGE advantages and large integrated user community deem this as the technology of choice. However, more highly discriminating subtyping methods have been sought to offer complementary analysis approaches.

Polymerization chain reactions (PCR) methods offer many technical advantages over the PFGE technique. PCR detects a small amount of a specific DNA sequence by amplifying it to levels that can be readily observed. PCR-based methods have become increasingly important to molecular typing efforts. These approaches include amplified fragment length polymorphism (AFLP), repetitive element polymorphisms-PCR, randomly amplified polymorphic DNA, and arbitrarily primed PCR The power of PCR-based methods is the ease with which they can be applied to many bacterial pathogens and their multilocus discrimination. However analysis of an entire genome is not possible with current PCR instruments. More discriminatory methods to provide molecular sub-typing have been sought.

It has recently been discovered that small polymorphic genomic regions, termed variable number tandem repeat VNTR sequences, provide a sensitive and reliable basis for molecular typing (7, 14, 15). VNTR are present in the genomes of most bacteria including E. coli. Many allelic states are observed in the VNTR among diverse strains and characteristic recurrence patterns are the basis of subtyping and identifying the strains. VNTR loci appear to be among the most diverse in bacterial genomes (21). As a result VNTRs appear to contain greater diversity and, hence, greater discriminatory capacity than any other type of molecular typing system.

The speed and efficiency of sub-typing bacteria is improved in multiple-locus VNTR analysis (MLVA). In MLVA, multiple markers are used to discriminate between related bacterial strains in a number of different isolates (Keim anthrax paper). U.S. Pat. No. 6,479,235 describes a multiplex technique that may be used for rapid, simultaneous analysis of DNA in multiple loci. MLVA may be used to resolve otherwise indistinguishable strain types and to phylogenetically define them relative to other close isolates (14, 15, 7).

DNA subtyping of the E. coli O157:H7 pathogenic strains by a MLVA system is a useful epidemiological approach. Because of the highly monomorphic molecular nature of E. coli, MLVA may be the only reasonable method with which to study the diversity, evolution, and molecular epidemiology of this pathogen. However, MLVA analysis requires identification of suitable marker DNA in the bacteria of interest and requires specific primers for amplifying the marker DNA.

### SUMMARY

Variable number tandem repeat (VNTR) sequences have been identified in the genome of certain E. coli strains. It has been discovered that the VNTR sequences exhibit length polymorphism at different loci. A sub-typing system based on multiloci size analysis of VNTR is the basis of the novel molecular sub-typing system of the present invention.

A molecular typing system is provided wherein VNTR sequences at a number of loci in an E. coli DNA sample are analyzed simultaneously and then evaluated for size. Discrete data is thereby generated that is characteristic of each sub-strain.

The small size of the VNTR relative to the whole genomic DNA makes it difficult to observe the sequences directly in a DNA sample with current technology. However, PCR methods are well known and may be used to amplify small loci containing the VNTR to amounts sufficient for size evaluation.

Accordingly, in preferred embodiments of the present invention, a molecular typing system is provided wherein multiloci containing VNTR are simultaneously amplified by PCR, preferably multiplex, and then separated by size. Size separation is preferably by gel or capillary electrophoresis. Tagged primers designed for each locus containing a VNTR sequence allow discrimination of loci and assignment of each amplified VNTR sequence to a genomic allele.

In an important aspect of the present invention, primers are provided for amplifying E. coli loci comprising VNTR. A representative sample of primers for amplifying loci from E. coli containing VNTR sequences include, but are not limited to the following primer pairs: SEQ ID No. and SEQ ID NO;

For use in the present sub-typing system, the primers comprise an observable indicator whereby an amplified loci containing a VNTR sequence may be identified, Preferably the indicator is a colored dye attached to one member of a primer pair, most preferably a fluorescent dye selected from the group consisting of HEX,

In certain embodiments of the present invention, multiplex cocktails containing two or more primers are provided for simultaneous amplification of multiloci containing VNTR in sample E. coli DNA. Suitable multiplex cocktails are exemplified by, but not limited to, the following primer sets having SEQ ID NO . . . .

In an important aspect of the present invention, kits are provided for use in sub-typing E. coli by PCR. In certain preferred embodiments, the kits comprise primers designed for E. coli loci containing a VNTR sequence. In certain other preferred embodiments, the kits comprise multiplex cocktails. The kits also comprise amplifying reagents for creating amplification conditions during an analysis in a PCR instrument. Generally the amplifying reagents comprise a polymerase, preferably taq polymerase, dntp selected from ATP, GTP, CTP and TPS and suitable salts and buffers to maintain amplification reaction conditions. In certain instances the kits may also comprise reference sample DNA. In certain other instances, the kits may comprise reagents and materials for allowing size separation and analysis of amplified products.

In yet another important aspect of the present invention, methods are provided for sub-typing an E. coli strain using PCR comprising the steps of:

(a) obtaining one or more primers specific for loci in an E. coli strain comprising VNTR sequence, said primer pair having an observable indicator,

(b) obtaining single-stranded sample DNA from the E. coli sample to be subtyped;

(c) combining said primers, said sample DNA and amplifying reagents under hybridizing and amplifying conditions in a PCR instrument to form amplicons comprising said primers and said marker DNA,

(d) separating the amplicons by size;

(e) evaluating the loci by observing the indicator in the separated amplicons and

(e) comparing said evaluation to an evaluation of a standard E. coli strain.

In an important aspect of the present invention, the VNTR sequences of the present invention are provided as a research tool for identifying novel molecular species, especially proteins, produced by the variable VNTR sequences present in rapidly evolving E. coli strains.

In yet another important aspect of the present invention, a method of producing discrete genetic data for an epidemiological database is provided. The data generated in the molecular sub-typing system of the present invention is in the form of discrete integral numbers about VNTR size and allelic location. A database containing this discrete information may be constructed worldwide over a long period of time. The database will be a powerful tool for containing the spread of disease.

### BRIEF DESCRIPTIONS OF THE FIGURES

FIG. 1. is a histogram of tandem repeats located in three E. coli genomes . . . .

FIG. 2. illustrates the alleles and diversity value for 30 polymorphic VNTR loci in 56 E. coli isolates.

FIG. 3 presents the genetic relationships among 56 E. coli isolates in a neighbor joining tree based upon analysis of 30 VNTR marker loci described in the present invention.

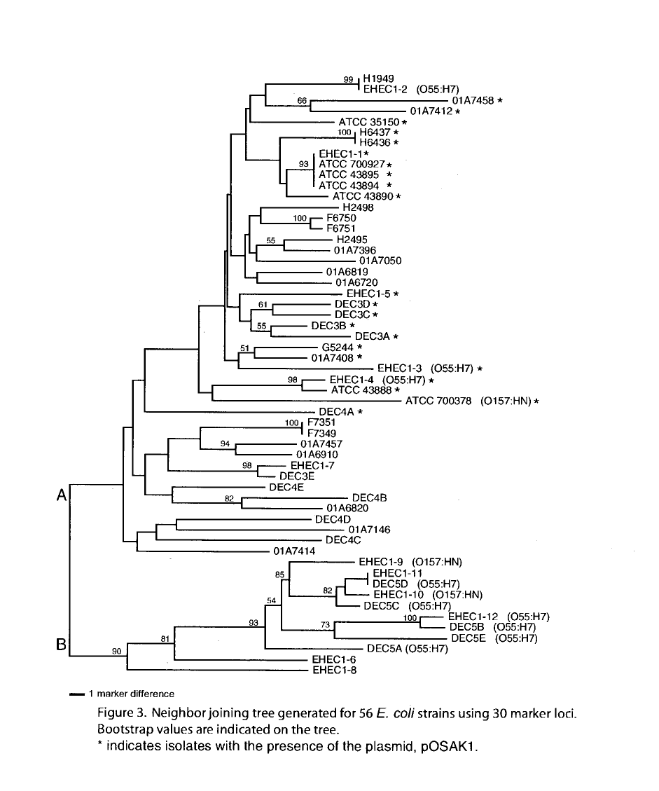

FIG. 4 is a photograph of an electrophoresis slab gel showing the separation pattern of amplified marker DNA from E. coli by size and colored primer. An E. coli strain may by sub-typed by comparing the pattern obtained in the gel with the pattern obtained with an E. coli strain of known type.

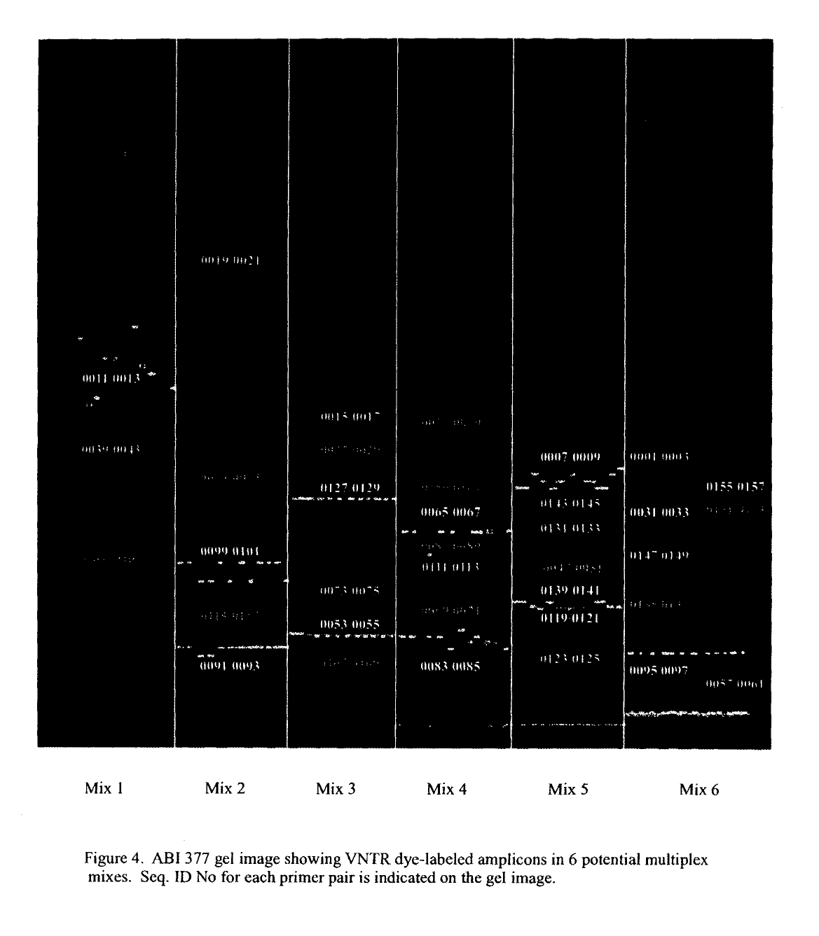

### DETAILS OF THE INVENTION

In one aspect, the present invention provides a molecular sub-typing system for E. coli based on analysis of VNTR loci. VNTR loci consist of short, repetitive sequence elements of a number of base pairs. Variation in the number of repeat units at a particular locus is responsible for the observed polymorphism observed at VNTR loci and is the basis of the present sub-typing system. Repeat arrays in an unknown E. coli strain are observed and compared to known strains. The VNTR locus repeat-size is easily defined, allowing the designation of specific alleles in a discrete, rather than continuous, data set. This is a great advantage for database results generated from multiple laboratories across several years.

VNTR loci have been identified in two E. coli whole genomic sequences and used to subtype E. coli O157:H7 strains. A representative sample of VNTR loci sequences according to the present invention include, but are not limited to SEQ ID numbers. 0163 to 0320, inclusive. It is to be understood that certain substitutions in these sequences occur naturally, but such substitutions do preclude the functionality of the VNTR loci for use in the present sub-typing system. Accordingly, VNTR loci functionally equivalent to the SEQ ID numbers 0163 to 0320 are intended to be included as members of the group.

It is anticipated that VNTR loci will be discovered in other E. coli and eventually will provide the basis for a global E. coli molecular sub-typing system. The present description is intended to provide details of a sub-typing system for E. coli O157:H7 that is exemplary of the system to be used for sub-typing other E. coli strains.

In a preferred embodiment of the present invention, the molecular sub-typing system comprises,

(a) primers for amplifying VNTR loci from E. coli DNA sample, said primers including an observable indicator;

(b) means for amplifying said primer and VNTR loci DNA to form amplicons;

(c) means for size-separating amplicons

(d) means for observing the indicator on said separated amplicons and

(e) means for calculating the VNTR repeat array in the E. coli DNA.

In an important aspect, primers are presented for amplifying VNTR loci from E. coli O157:H7 in PCR, preferably multiplex. A representative sample of primers that have been designed around these loci to amplify VNTR loci sequences according to the present invention include, but are not limited to SEQ ID NO's 0001 to 0162.inclusive.

In operation these primers are used in pairs selected from the group:

SEQ ID No. 0011+0013,

SEQ ID No. 0103+0105,

0035+0037,

0039+0043,

0091+0093,

0099+0101,

0115+0117,

0023+0025

0019+0021,

0053+0055,

0127+0129,

0107+0109,

0027+0029,

0073+0075,

0015+0017,

0083+0085,

0069+0071,

0047+0051,

0077+0079,

0111+0113,

0119+0121,

0065+0067,

0007+0009,

0087+0089,

0123+0125,

0139+0141,

0159+0161,

0057+0061,

0001+0003,

0031+0033,

0095+0097,

0131+0133,

0135+0137,

143+0145,

0147+0149,

0151+0153, and

0155+0157.

In the present method, amplification of the VNTR loci from E. coli results in amplicons comprising DNA of the primer pairs and the VNTR loci. Representative of amplicons comprising primers and VNTR loci from E. coli O157:H7 selected from the group consisting of SEQ ID NO. 0321 to SEQ ID NO. 0478 inclusive.

For use in sub-typing E. coli, the primers have an observable indicator. Preferably, the indicator is a dye attached to the primer. When amplified, the dye is incorporated into the amplicon and, after size separation of the amplicons, indicates the VNTR locus of each of the separated amplicons. The allelic array of VNTR is thus associated with discrete data that is characteristic of each E. coli strain and allows identification of strains.

Fluorescent dyes in commercial use are suitable indicators for use as indicators in the present sub-typing system. Preferred fluorescent dyes are HEX, FAM, NED, ROX available from Applied Biosystems (Foster City, Calif.) and dyes supplied by Beckman, Inc. (Fullerton, Calif.).

Preferred embodiments of the present invention are directed to MLVA methods of simultaneously analyzing multiple VNTR loci sequences. In preferred embodiments of the present invention, the PCF technique termed “multiplex” amplification methods are employed. In this technique, multiplex cocktails containing two or more labeled primer pairs are prepared and used for determining multiple VNTR loci in a sample DNA simultaneously. This technique generates large amount of data from a single amplification and thus provides efficiency and cost savings without loss of discriminatory power.

Multiplex cocktails for sub-typing E. coli O157:H7 are presented. The cocktails comprise primer sets selected from the group consisting of:

Set number one containing primers SEQ ID No. 0011 and 0013, SEQ ID No 0103 and 0105, SEQ ID No 0035 and 0037, SEQ ID No 0039 and 0043;

Set number two containing primers having seq. ID No.0091 and 0093, 0099 and 0101, 0115 and 0117, 0023 and 0025, 0019 and 0021;

Set number three having Seq. ID No 0053+0055, 0127+0129, 0107+0109, 0027+0029, 0073+0075, 0015+0017;

Set number four D No 0083+0085, 0069+0071, 0047+0051, 0077+0079, 0111+0113

Set number five Seq. ID No 0119+0121, 0065+0067, 0007+0009, 0087+0089, 0123+0125, 0139+0141; and

Set number six containing primers Seq. ID No 0159+0161, 0057+0061, 0001+0003

In an important aspect of the present invention, kits are provided for supplying sub-typing reagents needed to amplify VNTR loci in a PCR instrument. The kits supply primers or sets of primers for VNTR loci in the bacteria of interest. The kits also supply the necessary reagents for creating the hybridization and amplification conditions during a PCR run. Preferably the reagents comprise an amplifying agent, most preferably taq polymerase, dntp as building blocks, and salts and buffers for the reactions. The commercial availability of kits will encourage the development and use of the present E, coli molecular system. The ease of use of the kits and the increasing simplicity of the PCR technique will allow researchers and clinicians in even remote parts of the world to analyze infectious strains by the present sub-typing system. This will improve the containment of disease at the point of outbreak worldwide.

In a preferred embodiment of this aspect, kits are provided for sub-typing E. coli for use in PCR amplifications. In certain instances kits for sub-typing E. coli O157:H7 are. These kits comprise primers of the present invention for amplifying VNTR loci from this strain by PCR. In other preferred embodiments, kits comprise multiplex cocktails as described hereinabove are provided for multiplexing.

In an important aspect of the present invention methods are provided for sub-typing E. coli. The method comprises the steps of:

(a) obtaining one or more primers for amplifying loci comprising VNTR said primers having an observable indicator,

(b) obtaining single-stranded sample DNA from the E. coli sample to be subtyped;

(c) combining said primers, said sample DNA and amplifying reagents under hybridizing and amplifying conditions in a PCR instrument to form amplicons comprising said primers and said VNTR;

(d) separating the amplicons by size;

(e) evaluating numbers and sizes of separated amplicons and

(e) comparing said evaluation to an evaluation of amplicons obtained by PCR from a known E. coli strain.

The method may be modified for sub-typing a strain of interest by employing a primer specific for VNTR loci identified in known strains. In preferred embodiments the method may be used to sub-type E. coli O157:H7 by using the primers having sequence ID NO 0163 to 0320, inclusive. Any primers or multiplex cocktails capable of use for amplifying the VNTR loci having SEQ ID numbers. 0163 to 0320, inclusive may be may be used in the present method.

In preferred embodiments of the invention, amplicons are size-separated by gel electrophoresis or capillary electrophoresis.

In yet another important aspect of the invention, the sub-typing method may be used to produce discrete genetic data for an epidemiological database. The method generates information concerning VNTR arrays in certain alleles of E. coli. This data is provided in the form of discrete numbers that can be compared to numbers generated from analysis of known E. coli strains and sub-strains. A database of known strains will be compiled and identification of unknown strains from clinical isolates is made possible by comparison to known strains. The epidemiological value of this database for global control of diseases caused by bacterial infection is profound.

In yet another important aspect of the present invention, certain VNTR loci sequences are provided for use as research tools. It is known that certain E. coli strains are rapidly evolving and this is reflected in the variable polymorphism of the VNTR loci. The methods and means of the present invention may be used to identify and amplify these loci to study the molecules expressed.

The present invention may be better understood with reference to the accompanying examples that are intended for purposes of illustration only and should not be construed to limit the scope of the invention, as defined by the claims appended hereto.

### EXAMPLES

### Example 1

This Example illustrates the method of the present invention for molecular sub-typing of a sample DNA by multiplex.

DNA was prepared from a single colony of a pure culture as a simple whole-cell heat lysate from a single colony. This involves boiling a colony of E. coli in Tris-EDTA for 20 min and then removing the cellular debris through centrifugation. The remaining liquid contains a crude DNA extract that is suitable for use in this system.

All reagents used for PCR were obtained from Life Technologies, unless otherwise noted. PCR conditions for all mixes use 1U Platinum Taq, 1X PCR buffer, 2 mM MgCl₂ and 0.2 mM dNTPs final concentration in a total reaction volume of 10 ul. Primer concentrations for each multiplex mix are as follows: Mix 1 has primers pairs with Seq. ID No. 0011/0013, 0103/0105, 0035/0037, and 0039/0043 at final concentrations of 0.1, 0.6, 0.2, and 0.3 mM respectively; Mix 2 has primers pairs with Seq. ID No. 0091/0093, 0099/0101, 0115/0117, 0023/0025 and 0019/0021 at final concentrations of 0.05, 0.1, 0.1, 0.5, and 0.4 mM respectively; Mix 3 has primers pairs with Seq. ID No 0053/0055, 0127/0129, 0107/0109, 0027/0029, 0073/0075, and 0015/0017 at final concentrations of 0.1, 0.2, 0.1, 0.4, 0.05, and 0.3 mM respectively; Mix 4 has primers pairs with Seq. ID No 0083/0085, 0069/0071, 0047/0051, 0077/0079, and 0111/0113 at final concentrations of 0.1, 0.3, 0.2, 0.4, and 0.1 mM respectively; Mix 5 has primers pairs with Seq. ID No 0119/0121, 0065/0067, 0007/0009, 0087/0089, 0123/0125, and 0139/0141 at final concentrations of 0.2, 0.3, 0.2, 0.1, 0.1, and 0.6 mM respectively; and Mix 6 has primers pairs with Seq. ID No 0159/0161, 0057/0061, 0001/0003 at final concentrations of 0.2, 0.05, and 0.6 mM respectively. The remaining primers pairs with Seq. ID No, 0031/0033, 0095/0097, 0131/0133, 0135/0137, 143/0145, 0147/0149, 0151/0153, and 0155/0157 are not currently multiplexed, but are run under identical conditions to the above multiplex mixes with the exception that a final concentration of 0.2 mM is used for each primer. Future plans include incorporating these final markers into the existing multiplexes. To each 9 ul of master mix for the PCR reaction, 1 ul of a 1/10 dilution of the heat lysate DNA was added. Thermocycling parameters raised the PCR mixtures to an initial temperature of 94° C. for 5 min, with cycling of 94° C. for 20 sec, 65° C. for 20 sec, and 72° C. for 20 sec a total of 35 times with a final extension step of 72° C. for 5 min. PCR products were diluted five-fold prior to combining equally with ROX-labeled Map Marker 1000 (BioVentures, Inc.) custom size standard. Fluorescently labeled PCR product was visualized using polyacrylamide gel electrophoresis on a Perkin-Elmer Applied Biosystems 377 DNA sequencer. Fragment sizing was performed using GeneScan and Genotyper analysis software (Perkin-Elmer, ABI).

### Example 2

This Example illustrates the detection of VNTR sequences useful for sub-typing.

Tandem repeat structures were detected in the completed genomes of the K-12, EDL933 O157:H7, Sakai O157:H7 and in plasmids pO157 and pOSAK1 sequences obtained from the NCBI genome website. Repeats were found with the use of two software programs. Small (1 to 10 bp motif) perfect repeats were detected using SSR Finder (Gur-Arie et. al. 2000). Larger (>8 bp) perfect and imperfect repeats were found using GeneQuest (DNAstar software; LaserGene, Inc., Madison, Wis.). This program was also used to preliminarily determine if arrays were located in an ORF, while final confirmation was made by blasting the sequences against the annotated genome at the NCBI website server. Primers were designed around potential VNTRs using PrimerSelect (DNAstar software) or Oligo (ver. 6.52, Molecular Biology Insights, Inc.). Primers were designed with a Tm range of 68 to 72° C.

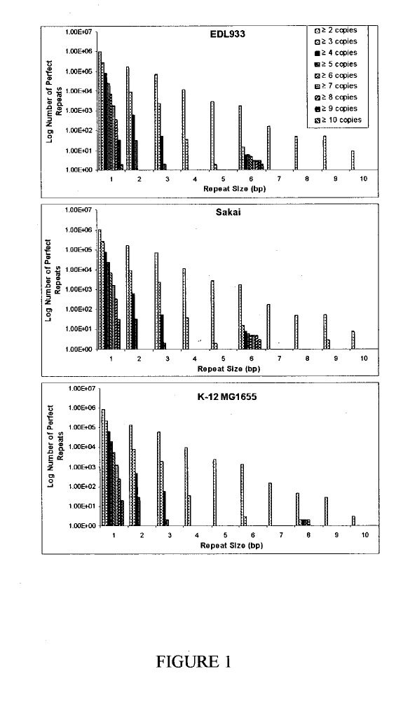

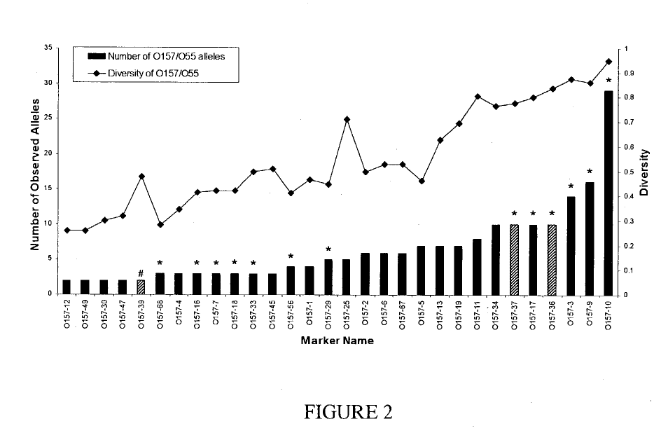

As illustrated in FIG. 1, there are thousands of potential VNTR loci in the E. coli genome. 67 potential VNTR loci were selected based upon the repeat size and copy number and were screened by PCR to maximize discrimination power and suitability for an electrophoretic assay. Of these, 37 generated robust PCR amplification and exhibited significant size variations strains. FIG. 2 illustrates the Locus O157-39 (#) was monomorphic, but useful as a presence-absence diagnostic marker for pOSAK1. Markers that contain a null-state allele in addition to fragment size variation are indicated with an asterisk (*). The three markers indicated with striped bars are located on plasmids.

These potential VNTR loci were screened for variability against 56 E. coli O157:H7/HN and O55:H7 strains (Table 1). Of the original 67 primer sets, 37 were chosen for use in the final analysis (Table 2).

### Example 3

This Example illustrates a scenario wherein the E. coli sub-typing system of the present invention allows rapid identification and containment of an infectious outbreak.

A food borne disease outbreak has occurred where food has been contaminated with pathogenic E. coli O157:H7. Public health, law enforcement or other agencies have provided the diagnostic laboratory a clinical E. coli isolate from a disease victim who ate the contaminated food. They would like to determine if the victim's bacterial isolate is the same subtype as is found in the contaminated food and an E. coli isolate from a particular food processing plant, or restaurant. Live cultures of each are provided to the diagnostic laboratory. A small portion of each culture is mixed with a small amount of an aqueous buffer and boiled for 10 to 20 minutes. This culture lysate is used as a source of DNA for PCR analysis of multiple variable number tandem repeat (VNTR) loci. A kit is provided containing primers and necessary amplification reagents. After reaction, the PCR products (amplicons) are separated by size via electrophoresis and detected by virtue of a fluorescent dye attached to one primer for each locus-specific primer pair. The number of sequence repeats at multiple VNTR loci is determined by estimation of the PCR amplicon size. These sizes represent a multiple locus genotype that will be compared to a standardized database of known strain genotypes and to the possible contamination source in the food processing plant, or restaurant. A positive strain identification will permit the plant or restaurant to remove the source of contaminated food and thus contain the spread of disease.

TABLE 1
Identification of E. coli Isolates
Analyzed by Multiplex PCR with the
Primers of the present invention.

| | | | |
|---|---|---|---|
| ID | Originating | | |
| Source | Lab | Serotype | Origin |

O157:H7/HN Isolates

| | | | | |
|---|---|---|---|---|
| 35150 | ATCC | O157:H7 | no data | no data |
| 43888 | ATCC | O157:H7 | no data | no data |
| 43890 | ATCC | O157:H7 | USA-CA | no data |
| 43895 | ATCC | O157:H7 | USA-OR | hamburger |
| 43894 | ATCC | O157:H7 | USA-MI | human feces |
| 700378 | ATCC | O157:HN | no data | human feces |
| 700927 | ATCC | O157:H7 | derived from | |
| | | | ATCC | 43895 |
| H6436 | CDC | O157:H7 | USA-WI | human |
| H6437 | CDC | O157:H7 | USA-WI | taco meat |
| F7349 | CDC | O157:H7 | USA-GA | human |
| F7351 | CDC | O157:H7 | USA-GA | human |
| F6751 | CDC | O157:H7 | USA-NY | no data |
| F6750 | CDC | O157:H7 | USA-NY | no data |
| H2495 | CDC | O157:H7 | USA-CT | Apple Cider |
| H2498 | CDC | O157:H7 | USA-CT | Apple Cider |
| G5244 | CDC | O157:H7 | Japan-Sakai | no data |
| H1949 | CDC | O157:H7 | USA-WA | restaurant standard |
| 01A6720 | CA Dept | O157:H7 | USA-CA | human |
| | Hlth | | | |
| 01A6819 | CA Dept | O157:H7 | USA-CA | human |
| | Hlth | | | |
| 01A6820 | CA Dept | O157:H7 | USA-CA | human |
| | Hlth | | | |
| 01A6910 | CA Dept | O157:H7 | USA-CA | human |
| | Hlth | | | |
| 01A7050 | CA Dept | O157:H7 | USA-CA | human |
| | Hlth | | | |
| 01A7412 | CA Dept | O157:H7 | USA-CA | human |
| | Hlth | | | |
| 01A7146 | CA Dept | O157:H7 | USA-CA | human |
| | Hlth | | | |
| 01A7396 | CA Dept | O157:H7 | USA-CA | human |
| | Hlth | | | |
| 01A7408 | CA Dept | O157:H7 | USA-CA | human |
| | Hlth | | | |
| 01A7414 | CA Dept | O157:H7 | USA-CA | human |
| | Hlth | | | |
| 01A7457 | CA Dept | O157:H7 | USA-CA | human |
| | Hlth | | | |
| 01A7458 | CA Dept | O157:H7 | USA-CA | human |
| | Hlth | | | |
| EHEC1 1 | STEC | O157:H7 | USA-OR | hamburger |
| | Center* | | | |
| EHEC1 5 | STEC Center | O157:H7 | USA-WA | human |
| EHEC1 6 | STEC Center | O157:H7 | Japan- | human |
| | | | Okayama | |
| EHEC1 7 | STEC Center | O157:H7 | USA-WA | human |
| EHEC1 8 | STEC Center | O157:H7 | USA-CA | human |
| EHEC1 9 | STEC Center | O157:HN | Germany | human (child) |
| EHEC1 10 | STEC Center | O157:HN | no data | human |
| EHEC1 11 | STEC Center | O157:H7 | USA-WA | human |
| DEC3A | STEC Center | O157:H7 | USA-WA | human |
| DEC3B | STEC Center | O157:H7 | USA-WA | human |
| DEC3C | STEC Center | O157:H7 | USA-NM | human |
| DEC3D | STEC Center | O157:H7 | USA-MI | human |
| DEC3E | STEC Center | O157:H7 | Canada | human |
| DEC4A | STEC Center | O157:H7 | Argentina | calf |
| DEC4B | STEC Center | O157:H7 | Denmark | human |
| DEC4C | STEC Center | O157:H7 | Egypt | buffalo |
| DEC4D | STEC Center | O157:H7 | Japan | calf |
| DEC4E | STEC Center | O157:H7 | Denmark | human |

O55:H7 Isolates

| | | | | |
|---|---|---|---|---|
| EHEC1 2 | STEC Center | O55:H7 | USA-WA | human |
| EHEC1 3 | STEC Center | O55:H7 | Sri Lanka | human |
| EHEC1 4 | STEC Center | O55:H7 | USA-MI | human |
| EHEC1 12 | STEC Center | O55:H7 | no data | meat |
| DEC5A | STEC Center | O55:H7 | USA-NY | human |
| DEC5B | STEC Center | O55:H7 | USA-FL | human |
| DEC5C | STEC Center | O55:H7 | USA-NJ | human |
| DEC5D | STEC Center | O55:H7 | Sri Lanka | human |
| DEC5E | STEC Center | O55:H7 | Iran | human |

*http://www.shigatox.net

TABLE 2
VNTR Locus PCR Primers.

| | | |
|---|---|---|
| Marker¹ | Dye | Forward Primer (5′ to 3′) |
| | Dye | Reverse Primer (5′ to 3′) |

| | |
|---|---|
| Multiplex 1 | |
| Ned | |
| Seq. ID. No. 0013 GGCGGTAAGGACAACGGGGTGTTTGAATTG | Seq. ID. No. 0011 |
| GAACAACCTAAAACCCGCCTCGCCATCG | Seq. ID. No. 0013 |
| GCGCTGGTTTAGCCATCGCCTTCTTCC | Seq. ID. No. 0035 |
| Hex | |
| GTGTCAGGTGAGCTACAGCCCGCTTACGCTC | Seq. ID. No. 0037 |
| CAGCCTCCTGCAAACTTTACTGTTCATTTCTACAGTCTC | Seq. ID. No. 0077 |
| Fam | |
| GGATCTGTCTGTATCATCATTGAATGAACAACCCATTTC | Seq. ID. No. 0079 |
| Hex | |
| GACAAGGTTCTGGCGTGTTACCAACGG | Seq. ID. No. 0103 |
| GTTACAACTCACCTGCGAATTTTTTTAAGTCCC | Seq. ID. No. 0105 |
| Multiplex 2 | |
| Fam | |
| GATAACATGTCCGGCAAATATTCATTCCCTGAGCA | Seq. ID. No. 0019 |
| GTTTCGCGAATTTTGACAGTTTTTGCATCCTGATC | Seq. ID. No. 0021 |
| Hex | |
| GTCTTCATATTGTTTGCGATGTCCCTGATGAACTTATTGA | Seq. ID. No. 0023 |
| GTCCAGACGCCAGTGCAGCTTATTCTCCACG | Seq. ID. No. 0025 |
| Ned | |
| GTTGCCGACCCACAGCGATACGCCAT | Seq. ID. No. 0091 |
| AGCTGATTGCCAGATCGCTTTGCTCCAGAG | Seq. ID. No. 0093 |
| Ned | |
| GTGAAGGATAAGCTGCATTTGTCAGTGATGTCCGAAG | Seq. ID. No. 0099 |
| GCCTGACGCTAAAGATAAAGAAGAAAGCGTCGCG | Seq. ID. No. 0101 |
| Hex | |
| GGGTTTGTTTTCAGTGAAGTATTCGCCAAGGTTC | Seq. ID. No. 0115c |
| GATGTCGAAATGGAAGATTACTCAACATACTGCTTCTC | Seq. ID. No. 0117 |
| Multiplex 3 | |
| Fam | |
| GCCAGATAAACATCCAGCAGGTCGAACGTCC | Seq. ID. No. 0015 |
| GACTCTGCGGCAATATGGCGTCTTTAGTATCTCCTG | Seq. ID. No. 0017 |
| Hex | |
| GGGGCGATCCCACCCTCCATCCTG | Seq. ID. No. 0027 |
| GAGCGGCAATTGTAATCCGGTGGCTTCC | Seq. ID. No. 0029 |
| Ned | |
| GGCATCAATAAAAGGTAAGCCAAGTTTCGCCG | Seq. ID. No. 0053 |
| GCATCCTGAACCAACCTGGGTATGCTGC | Seq. ID. No. 0055 |
| Fam | |
| GACTGGCGATGAAGAGCGTTTTAATGAGTTTATCAGTGA | Seq. ID. No. 0073 |
| GAATGCGCTGTTCCCCTTCTTCCCTTCC | Seq. ID. No. 0075 |
| Hex | |
| GGCGTCCTTCATCGGCCTGTCCGTTAAAC | Seq. ID. No. 0107 |
| GCCGCTGAAAGCCCACACCATGC | Seq. ID. No. 0109 |
| Ned | |
| GTTCTTCATACAGCGTCCACGTCGGGCCT | Seq. ID. No. 0127 |
| GACTGGGAGCCATCATTACTTACGCAGCTTGAAC | Seq. ID. No. 0129 |
| Multiplex 4 | |
| Hex | |
| GACCGGCAATCATCGGGCCAACCA | Seq. ID. No. 0047 |
| GATGCTGGAAAAACTGATGCAGACTCGCGT | Seq. ID. No. 0051 |
| GCAGTTGCTCGGTTTTAACATTGCAGTGATGAC | Seq. ID. No. 0069 |
| Hex | |
| GGAAATGGTTTACATGAGTTTGACGATGGCGATC | Seq. ID. No. 0071 |
| GCAGTGATCATTATTAGCACCGCTTTCTGGATGTTC | Seq. ID. No. 0077 |
| Hex | |
| GGGGCAGGGAATAAGGCCACCTGTTAAGC | Seq. ID. No. 0079 |
| Ned | |
| GCCGGAGGAGGGTGATGAGCGGTTATATTTAGTG | Seq. ID. No. 0083 |
| GCGCTGAAAAGACATTCTCTGTTTGGTTTACACGAC | Seq. ID. No. 0085 |
| Fam | |
| GCCGCCCCTTACATTACGCGGACATTC | Seq. ID. No. 0111 |
| GCAGGAGAACAACAAAACAGACAGTAATCAGAGCAGC | Seq. ID. No. 0113 |
| Multiplex 5 | |
| Ned | |
| GGGCCAGCCGCTGTACCGGGGA | Seq. ID. No. 0007 |
| GTATGATGAAACGCTGACGGCGCTGGATG | Seq. ID. No. 0009 |
| Ned | |
| GTCGCTGATAATATTCTCTTTTCGTCATCCCACTGTTAC | Seq. ID. No. 0065 |
| AATACGGTATTGCCATCGGCTCCAAAAAGTTTATC | Seq. ID. No. 0067 |
| Hex | |
| GCTCTCCATGGTATCTTCTGACCCAGGGGTATCTA | Seq. ID. No. 0087 |
| GAAAGTTTCATCGGGGGCTGGCTACGGTCTTA | Seq. ID. No. 0089 |
| Ned | |
| GTTTCGGGTGAATAGAGGGCGCTTTTCTCGTTA | Seq. ID. No. 0119 |
| GTTCCTCACCAATATTGAAAACACGGCGTAGCAAAAAG | Seq. ID. No. 0121 |
| Fam | |
| GCCTGCGGCTGGGCAAATTCGTTCC | Seq. ID. No. 0123 |
| GATGCTCGCCTGATCGACAACAAAATGGTCG | Seq. ID. No. 0125 |
| Fam | |
| AACACTTTGTTCCACAAGAAAATTGTCAGGG | Seq. ID. No. 0139 |
| ATTATGTGCATAAAATTGGCATTGCTCTTTT | Seq. ID. No. 0141 |
| Multiplex 6 | |
| Fam | |
| GAGGGATTGTTACCTTGGTCTCAAAACAATGAAAGG | Seq. ID. No. 0001 |
| GTTCCAGCCCCTTCAACCTTAGCTTATTCTGGCTC | Seq. ID. No. 0003 |
| Fam | |
| GCAGCAAACGCCACAGTACCCATGCC | Seq. ID. No. 0057 |
| GTAGGTCATCTGCCGTGGTTCGAGCGCT | Seq. ID. No. 0061 |
| Hex | |
| GAAAATCCGGCGACGGTTGCCAGACTC | Seq. ID. No. 0159 |
| GCGGGAGCGGGAAAGACTGCGGA | Seq. ID. No. 0161 |

Eight loci polymorphic outside O157:H7 (not multiplexed)

| | |
|---|---|
| Ned | |
| GCTGTTCCCGTTCTTTGGCTTTACCGCC | Seq. ID. No. 0031 |
| GCGTTACGCCGCAGAACCCACCTGC | Seq. ID. No. 0033 |
| Ned | |
| GCCGAAAAACGATGCAGCTGACTTAGGCG | Seq. ID. No. 0095 |
| GACATTTCTGCCCGGGGGTTTGTTTATTTCTGC | Seq. ID. No. 0097 |
| Fam | |
| GCCCGCCGGGCCGATGACC | Seq. ID. No. 0131 |
| GGCGGCGTGGGGGATTATTGCCC | Seq. ID. No. 0133 |
| Hex | |
| GGGACTGGATATTGTGCAGGGTTCAGCAGG | Seq. ID. No. 0135 |
| GGGCCGGGCAGCGCAAGGTCC | Seq. ID. No. 0137 |
| Fam | |
| GCGGCGCATTAGCGTCGTATCAGGC | Seq. ID. No. 0143 |
| CAGTTTGGCCATGCGTCTGGGGTGAC | Seq. ID. No. 0145 |
| Fam | |
| GACTGAGGCTGTCATCTCGAAAGAGGGCATTCT | Seq. ID. No. 0147 |
| GCGCTGGGAGGTGTCGCTCAGATGG | Seq. ID. No. 0149 |
| Hex | |
| GTTTGCTGTAGCCCAGGCCGTTGATCTTCTTC | Seq. ID. No. 0151 |
| GTTCCGGCGGCGAAAGTTTCCTCGTTAG | Seq. ID. No. 0153 |
| Ned | |
| GACTTACTCAGCGCCGCCAACGAAGTCC | Seq. ID. No. 0155 |
| GCACCGCACGTTTCTGAAAAAGCGTCTACT | Seq. ID. No. 0157 |

¹Primer sets are arranged by multiplex PCR cocktails.

TABLE 3
VNTR Locus Attributes.

| | | | Features of repeat | |
|---|---|---|---|---|
| | Array² in EDL-933 | | Location of 5’ | |
| | ORF Identity | | Location | |
| | (in Sakai | | In | |
| Marker¹ | if different) | | EDL-933 | end of array |
| Multiplex 1 | | | | |
| O157-3 | 6 × 9 | OI #7 | 271423 | hypothetical |
| | | | | protein |
| O157-9 | 6 × 11 (6 × 10) | OI #108 | 3557714 | not in an ORF |
| O157-10 | 6 × 17.7 | OI #108 | 3559120 | hypothetical |
| | (6 × 25.7) | | | protein |
| O157-34 | 18 × 10 (18 × 9) | — | 5361545 | yjgL |
| Multiplex 2 | | | | |
| O157-5 | 56 × 2.2 | OI #67 | 2103941 | putative |
| | | | | BigA-like |
| | | | | protein |
| O157-6 | 8 × 4 | OI #64 | 2036603 | H repeat- |
| | | | | associated |
| | | | | protein of |
| | | | | Rhs element |
| O157-30 | 9 × 3 | OI #167 | 5197093 | putative |
| | | | | histidine |
| | | | | kinase |
| O157-33 | 16 × 3 | — | 5325245 | not in an ORF |
| O157-39 | 3 × 4 | pOSAK1 | 1603 | hypothetical |
| | | | | protein |
| Multiplex 3 | | | | |
| O157-4 | 33 × 2.3 | — | 1770140 | not in an ORF |
| O157-7 | 62 × 2.2 | — | 2716203 | fliI |
| O157-12 | 29 × 2 | OI #134 | 4360214 | putative ATP- |
| | | | | dependent |
| | | | | DNA helicase |
| O157-18 | 25 × 2.4 | — | 5462817 | hypothetical |
| | | | | protein |
| O157-36 | 7 × 10 | pO157 | 54348 | not in an ORF |
| O157-49 | 28 × 2 | OI #7 | 258805 | hypothetical |
| | | | | protein |
| Multiplex 4 | | | | |
| O157-11 | 6 × 5.5 | — | 4850327 | hemX |
| O157-17 | 6 × 6 (6 × 8) | OI #174 | 5456065 | hypothetical |
| | | | | protein |
| O157-19 | 6 × 6 (6 × 4) | — | 2932247 | hypothetical |
| | | | | protein |
| O157-25 | 6 × 5 (6 × 4) | — | 1605820 | not in an ORF |
| O157-37 | 6 × 7 | pO157 | 46468 | hypothetical |
| | | | | protein |
| Multiplex 5 | | | | |
| O157-2 | 12 × 2 | OI #7 | 252309 | putative |
| | | | | protease |
| O157-16 | 21 × 2 | OI #172 | 5385681 | hypothetical |
| | | | | protein |
| O157-29 | 6 × 3.5, 3 × 4 | on LEE, | | 4669380 tir |
| | | OI #148 | | |
| O157-45 | 5 × 4 | — | 46552 | not in an ORF |
| O157-47 | 7 × 2 | OI #4 | 152500 | not in an ORF |
| O157-56 | 5 × 3 | OI #55 | 1785903 | hypothetical |
| | | | | protein |
| Multiplex 6 | | | | |
| O157-1 | 15 × 4 | — | 64022 | hypothetical |
| | | | | protein |
| O157-13 | 9 × 4 | — | 4499709 | yhjN |
| O157-68 | 6 × 3 | OI #79 | 2781280 | unknown |
| | | | | prophage |
| | | | | CP-933U |
| | | | | protein |

Eight loci polymorphic outside O157:H7 (not multiplexed)

| | | | | |
|---|---|---|---|---|
| O157-8 | 19 × 2 | — | 3367638 | not in an ORF |
| O157-31 | 9 × 2, 8 × 2 | — | 5257006 | mopA |
| O157-50 | 9 × 2 | OI #7 | 248634 | putative |
| | | | | macrophage |
| | | | | toxin |
| O157-5 | 19 × 2 | OI #7 | 267217 | Rhs protein |
| O157-57 | 6 × 3 | — | 1304626 | appA |
| O157-58 | 8 × 2.3 | — | 1322683 | torA |
| O157-63 | 6 × 3 | — | 474840 | sbcC |
| O157-64 | 6 × 3 | — | 4253747 | 50S ribosomal |
| | | | | subunit |
| | | | | protein L23 |

¹Primer sets are arranged by multiplex PCR cocktails.
²Array nomenclature

### REFERENCES

1. Belongia, e. A., M. T. Osterholm, J. T. Solder, D. A. Ammend, J. E. Braun, and K. L. MacDonald. 1993. Transmission of Escherichia coli O157:H7 infection in Minnesota child daycare facilities. JAMA. 269:883-888.

2. Bokete, T., T. Whittham, R. Wilson, C. Clausen, C. O'Callahan, S. Moseley, T. Fritsche, and P. Tarr. 1997. Genetic and phenotypic analysis of Escherichia coli with enteropathogenic characteristics isolated from Seattle children. 175:1382-1389.

3. Centers for Disease Control and Prevention. 2002. Multistate outbreak of Escherichia coli O157:H7 infections associated with eating ground beef—United States June-July 2002. MMWR. 51:637-639.

4. Centers for Disease Control and Prevention. 1997. Escherichia coli O157:H7 infections associated with eating a nationally commercial brand of frozen ground beef patties and burgers—Colorado, 1997. MMWR. 46:777-778.

5. Centers for Disease Control and Prevention. 1996. Lake-associated outbreak of Escherichia coli O157:H7—Illinois 1995. MMWR. 45:437-439.

6. Clark, C., J. Beltrame, and P. Manning. 1991. The oac gene encoding a lipopolysaccharide O-antigen acetylase maps adjacent to the integrase-encoding gene on the genome of Shigella flexneri bacteriophage Sf6. Gene. 107:43-52.

7. Farlow, J., K. L. Smith, J. Wong, M. Abrams, M. Lytle, and P. Keim. 2001. Francisella tularensis strain typing using multiple-locus, variable-number tandem repeat analysis. J. Clin. Microbiol. 39:3186-3192.

8. Feng, P. 1995. Escherichia coli serotype O157:H7: novel vehicles of infection and emergence of phenotypic variants. Emerging Infect Dis. 1:47-52.

9. Grimm, L. M., M. Goldoft, J. Kobayashi, J. H. Lewis, D. Alfi, A. M. Perdichizzi, P. I. Tarr, J. E. Ongerth, S. L. Moseley, and M. Samadpour. 1995. Molecular epidemiology of a fast-food restaurant-associated outbreak of Escherichia coli O157:H7 in Washington State. J Clin Microbiol. 33:2155-2158.

10. Gur-Arie, R., C. J. Cohen, V. Eitan, L. Shelef, E. M. Hallerman, and V. Kashi. 2000. Simple sequence repeats in Escherichia coli: abundance, distribution, composition, and polymorphism. Genome Res. 10:62-71.

11. Ito, T., K Hiramasu, V. Ohshita, and T. Yokota. 1993. Mutations in the rftT gene are responsible for the Ogawa to Inaba serotype conversion in Vibrio cholerae O1. Microbiol Immunol. 37:281-288.

12. Keene, W., J. McAnulty, F. Hoesly, L. J. Williams, K. Hedberg, G. Oxman, T. Barrett, M. Pfaller, and D. Fleming. 1994. A swimming-associated outbreak of hemorrhagic colitis caused by Escherichia coli O157:H7 and Shigella sonnei. N Engl J Med 331:579-584.

13. Keenleyside, W., and C. Whitfield. 1995. Lateral transfer of rfb genes: a mobilizable ColE1-type plasmid carries the rfb_{O:54} (O:54 antigen biosynthesis) gene cluster from Salmonella enterica serovar borreze. J Bacteriol. 177:5247-5253.

14. Keim, P., L. B. Price, A. M. Klevytska, K. L. Smith, J. M. Schupp, R. Okinaka, P. J. Jackson, and M. E. Hugh-Jones. 2000. Multiple-locus variable-number tandem repeat analysis reveals genetic relationships within Bacillus anthracis. J. Bacteriol. 182:2928-2936.

15. Klevytska, A. M., L. B. Price, J. M. Schupp, P. L. Worsham, J. Wong, and P. Keim. 2001. Identification and characterization of variable-number tandem repeats in the Yersinia pestis genome. J. Clin. Microbiol. 39: 3179-3185.

16. Metzgar, D. E. Thomas, C. Davis, D. Field, and C. Wills. 2001. The microsatellites of Escherichia coli: rapidly evolving repetitive DNAs in a non-pathogenic eukaryote. Mol Microbiol. 39:183-190.

17. Perna, N. T., G. Plunkett III, V. Burland, B. Mau, J. D. Glasner, D. J. Rose, G. F. Mayhew, P. S. Evans, J. Gregor, H. A. Kirkpatrick, G. Posfai, J. Hackett, S. Klink, A. Boutin, V. Shao, L. Miller, E. J. Grotbeck, N. W. Davis, A. Lim, E. Dimalanta, K. Potamousis, J. Apodaca, T. S. Anantharaman, J. Lin, G. Yen, D.C. Schwartz, R. A. Welch and F. R. Blattner. 2001. Genome sequence of enterohaemorrhagic Escherichia coli O157:H7. Nature 409:529-533.

18. Riley, L. W., R. S. Remis, S. D. Helgerson, H. B. McGee, J. G. Wells, B. R. Davis, R. J. Herbert, E. S. Olcott, L. M. Johnson, N. T. Hargrett, P. A. Blake, and M. L. Cohen. 1983. Hemorrhagic colitis associated with a rare Escherichia coli serotype. N Engl J Med 308:681-685.

19. Swaminathan, B., T. J. Barrett, S. B. Hunter, R. V. Tauxe, and the CDC Pulse Net Task Force. 2001. PulseNet: the molecular subtyping network for foodborne bacterial disease surveillance, United States. Emerging Infect Dis. 7:382-389.

20. Swerdlow, D., B. Woodruff, and R. Brady. 1992. A waterborne outbreak in Mossouri of Escherichia coli O157:H7 associated with bloody diarrhea andcdeath. Ann Intern Med. 117:812-819.

21. van Belkum, A., S. Sherer, L. van Alphen, and H. Verbrugh. 1998. sequence DNA repeats in prokaryotic genomes. Microbiol Mol Biol Rev. 62:275-293.

22. Weir, B. S. 1990. Genetic Data Analysis: Methods for Discrete Population Genetic Data analysis. Sinauer Associates, Inc. Publishers, Sunderland, Mass.

23. Whittam, T. S. 1998. Evolution of Escherichia coli O157:H7 and other shiga toxin-producing E. coli strains, p. 195-209. In J. B. Kaper and A. D. O'Brien (ed.), Escherichia coli O157:H7 and other shiga toxin-producing E. coli strains, 1^{st} ed. American Society for Microbiology, Washington, D.C.

24. Whittam, T. S. 1996. Genetic variation and evolutionary processes in natural populations of Escherichia coli, p. 2708-2720. In F. C. Neidhardt (ed.), Escherichia coli and Salmonella: cellular and molecular biology, 2^{nd} ed. American Society for Micriobiology, Washington, D.C.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

We claim:

1. A molecular sub-typing system for E. coli comprising observing and recording VNTR repeat arrays in an E. coli DNA sample.

2. A molecular sub-typing system of claim 1 comprising:
(a) primers for amplifying VNTR loci from E. coli DNA sample, said primers including an observable indicator,
(b) means for amplifying said primer and VNTR loci DNA to form amplicons;
(c) means for size-separating amplicons formed from the primer and VNTR loci;
(d) means for observing the indicator on said separated amplicons and
(e) means for calculating the VNTR repeat array in the E. coli DNA.

3. VNTR loci for sub-typing E. coli O157:H7, comprising sequence selected from the group consisting of SEQ ID numbers. 0163 to 0320, inclusive

4. A locus of claim 3 amplified by PCR.

5. Primers for amplifying a locus of claim 3

6. Primers of claim 4 selected from the group consisting of SEQ ID NO's 0001 to 0162.

7. Amplicon comprising a primer of claim 5 and a locus comprising a VNTR sequence from E. coli O157:H7 selected from the group consisting of SEQ ID NO. 0321 to SEQ ID NO. 0478

8. Primer pairs for amplifying loci of claim 3 selected from the group consisting of Seq. ID No. 0011+0013, 0103+0105, 0035+0037, 0039+0043, 0091+0093, 0099+0101, 0115+0117, 0023+0025 0019+0021, 0053+0055, 0127+0129, 0107+0109, 0027+0029, 0073+0075, 0015+0017, 0083+0085, 0069+0071, 0047+0051, 0077+0079, 0111+0113, 0119+0121, 0065+0067, 0007+0009, 0087+0089, 0123+0125, 0139+0141, 0159+0161, 0057+0061, 0001+0003, 0031+0033, 0095+0097, 0131+0133, 0135+0137, 143+0145, 0147+0149, 0151+0153, and 0155+0157.

9. Primers of claim 8 wherein one member of said pair has an observable indicator.

10. Primers of claim 9 wherein said indicator is a fluorescent dye.

11. Primers of claim 10 wherein said fluorescent dye is HEX, FAM, NED or ROX.

12. Multiplex cocktails for multiplex amplification of a locus of claim 3 comprising two or more primers of claim 9.

13. A multiplex cocktail of claim 12 comprising a primer set selected from the group consisting of:
Set number one containing primers SEQ ID No. 0011 and 0013, SEQ ID No 0103 and 0105, SEQ ID No 0035 and 0037, SEQ ID No 0039 and 0043;
Set number two containing primers having seq. ID No.0091 and 0093, 0099 and 0101, 0115 and 0117, 0023 and 0025, 0019 and 0021;
Set number three having Seq. ID No 0053+0055, 0127+0129, 0107+0109, 0027+0029, 0073+0075, 0015+0017;
Set number four D No 0083+0085, 0069+0071, 0047+0051, 0077+0079, 0111+0113
Set number five Seq. ID No 0119+0121, 0065+0067, 0007+0009, 0087+0089, 0123+0125, 0139+0141; and
Set number six containing primers Seq. ID No 0159+0161, 0057+0061, 0001+0003

14. Kits for molecular sub-typing of E. coli by PCR comprising:
(a) primers for VNTR loci in E. coli
(b) amplifying reagents for maintaining hybridization and amplification conditions in a PCR instrument with DNA from an E. coli strain.

15. Kits for molecular sub-typing E. coli O157:H7 strains by PCR comprising:
(a) one or more primers of claim 9; and
(b) amplifying reagents for maintaining hybridization and amplification conditions in a PCR instrument with DNA from an E. coli O157:H7 strain.

16. Kits for molecular sub-typing E. coli O157:H7 strains by multiplex comprising a multiplex cocktail of claim 13 and amplifying reagents for maintaining hybridization and amplification conditions in a multiplex instrument with DNA from an E. coli O157:H7 strain.

17. A method for sub-typing an E. coli strain comprising the steps of:
(a) obtaining one or more primers for amplifying loci comprising VNTR said primers having an observable indicator,
(b) obtaining single-stranded sample DNA from the E. coli sample to be subtyped;
(c) combining said primers, said sample DNA and amplifying reagents under hybridizing and amplifying conditions in a PCR instrument to form amplicons comprising said primers and said VNTR;
(d) separating the amplicons by size;
(e) evaluating numbers and sizes of separated amplicons and
(e) comparing said evaluation to an evaluation of amplicons obtained by PCR from a known E. coli strain.

18. A method of claim 17 for sub-typing an E. coli O157:H7 strain by multiplex wherein said primers are designed to amplify VNTR loci in E. coli O157:H7.

19. The method of claim 17 wherein said amplicons are separated by gel electrophoresis or capillary electrophoresis.

20. A method claim 17 for producing discrete genetic data for an epidemiological database

21. The VNTR sequences of claim 3 as a research tool.
